(19) United States
(12) Patent Application Publication
(10) Pub. No.: US 2026/0191436 A1
(43) Pub. Date: Jul. 9, 2026

(54) NON-INVASIVE ANALYTE SENSOR

(71) Applicant: Roche Diabetes Care GmbH, Mannheim (DE)

(72) Inventors: Alexander Steck, Hirschberg (DE); Kirill Sliozberg, Mannheim (DE)

(21) Appl. No.: 19/557,395

(22) Filed: Mar. 5, 2026

Related U.S. Application Data
(63) Continuation of application No. PCT/EP2024/075165, filed on Sep. 10, 2024.

(30) Foreign Application Priority Data
Sep. 13, 2023 (EP) 23197217.5

(51) Int. Cl. A61B 5/145; A61B 5/1486

(52) U.S. Cl. CPC A61B 5/14532; A61B 5/1486

(57) ABSTRACT

A non-invasive analyte sensor with at least two electrodes, each having a skin contacting surface. At least one of the electrodes is a detection electrode. The detection electrode includes at least one analyte-responsive layer and at least one conversion layer. At least one other of the electrodes is configured as an auxiliary electrode configured as either a counter, or a combined counter-reference electrode.

### CROSS REFERENCE TO RELATED APPLICATIONS

This is a continuation of application serial no. PCT/EP2024/075165 filed Sep. 10, 2024, which claims priority to EP 23 197 217.5 filed Sep. 13, 2023, the disclosures of both of which are hereby incorporated herein by reference.

### BACKGROUND

### Technical Field

The present application relates to non-invasive analyte sensors.

### Background Art

Non-invasive continuous monitoring systems for detection of an analyte such as for the detection of glucose are generally known, e.g., from U.S. Pat. No. 10,034,625B1, EP3641633A1, US20180368743A1 or U.S. Pat. No. 11,609,163B2. Such systems may be worn on-skin such as may be applied to the skin by using at least one patch or may be worn such as a wristwatch.

For example, non-invasive monitoring systems for detection of an analyte are known which detect the analyte while the analyte is not being actively withdrawn from the detection volume. However, these non-invasive monitoring systems suffer with the problem of slow resorption of glucose by the skin, so that the system would show the accumulated glucose concentration, which does not correspond to the actual glucose levels in, e.g., the interstitial fluid (ISF).

Moreover, equilibrium sensors for invasive glucose monitoring are known. However, an equilibrium sensor does not actively consume glucose but can only work under conditions, where a bulk glucose concentration actively changes, e.g., in-vivo or in some reactors, where the glucose levels may be monitored and are varied extrinsically. Correspondingly, such kind of arrangements are not suitable for a non-invasive on-skin sensor, which measures glucose levels in the bodily leachates, such as sweat.

### SUMMARY

The present disclosure concerns a non-invasive analyte sensor, a non-invasive continuous monitoring system for on-skin wearing comprising at least one non-invasive analyte sensor, a method for determining a concentration of an analyte in a sample using a non-invasive continuous monitoring system and a computer program. The method and devices according to the present disclosure may be used for detecting at least one analyte present in bodily leachates. In particular the method and devices are applied in the field of detecting one or more analytes such as glucose or other analytes in bodily leachates, such as sweat, both in the field of professional diagnostics, in the field of hospital point of care, in the field of personal care and in the field of home monitoring. However, other fields of application are feasible.

The present disclosure provides a non-invasive analyte sensor, a non-invasive continuous monitoring system for on-skin wearing and a method for determining a concentration of an analyte in a sample, which at least partially avoid the shortcomings of known devices and methods of this kind and which at least partially address the above-mentioned challenges. Specifically, devices and methods shall be provided which allow for a precise and non-invasive determination of an analyte concentration.

This problem is addressed by a non-invasive analyte sensor, a non-invasive continuous monitoring system for on-skin wearing comprising at least one non-invasive analyte sensor, a method for determining a concentration of an analyte in a sample using a non-invasive continuous monitoring system and a computer program with the features of the independent claims. Advantageous embodiments which might be realized in an isolated fashion or in any arbitrary combinations are listed in the dependent claims as well as throughout the specification.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

Further, as used in the following, the terms “preferably”, “more preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

In a first aspect of the present disclosure, a non-invasive analyte sensor is disclosed. The non-invasive analyte sensor comprises at least two electrodes, each having a skin contacting surface. At least one of the electrodes is a detection electrode. The detection electrode comprises at least one analyte-responsive layer and at least one conversion layer. At least one other of the electrodes is configured as an auxiliary electrode configured as either a counter, or a combined counter-reference electrode.

The term “analyte” as used herein is a broad term and is to be given its ordinary and customary meaning to a person of ordinary skill in the art and is not to be limited to a special or customized meaning. The term specifically may refer, without limitation, to an arbitrary element, component or compound which may be present in a fluid, specifically in a bodily fluid, and the concentration of which may be of interest for a user. Specifically, the analyte may be or may comprise an arbitrary chemical substance or chemical compound which may take part in the metabolism of the user, such as at least one metabolite. As an example, the at least one analyte may be selected from the group consisting of glucose, lactate, ascorbate and any other analyte, for which an analyte-responsive layer can be designed and which can be followingly converted by a suitable conversion layer. For example, the analyte is glucose. Additionally or alternatively, however, other types of analytes may be determined and/or any combination of analytes may be determined. The term “fluid” as used herein is a broad term and is to be given its ordinary and customary meaning to a person of ordinary skill in the art and is not to be limited to a special or customized meaning. The term specifically may refer, without limitation, to biofluids of interest known to comprise or suspected to comprise the analyte. The fluid may be a bodily fluid, for example, a bodily leachate, such as sweat, tears, saliva. The bodily fluid generally may be contained in a body tissue. The detection of the at least one analyte in the bodily fluid may preferably be determined on skin. The analyte sensor may be an on skin sensor. The term “on skin sensor” as used herein is a broad term and is to be given its ordinary and customary meaning to a person of ordinary skill in the art and is not to be limited to a special or customized meaning. The term specifically may refer, without limitation, to a sensor which is configured for analyte detection on the living tissue at the skin surface.

The term “sensor” as used herein is a broad term and is to be given its ordinary and customary meaning to a person of ordinary skill in the art and is not to be limited to a special or customized meaning. The term specifically may refer, without limitation, to an arbitrary element or device configured for detecting at least one condition or for measuring at least one measurement variable. The term “analyte sensor” as used herein is a broad term and is to be given its ordinary and customary meaning to a person of ordinary skill in the art and is not to be limited to a special or customized meaning. The term specifically may refer, without limitation, to a sensor configured for detecting quantitatively or qualitatively at least one analyte. The non-invasive analyte sensor may be configured for continuous analyte monitoring.

The term “non-invasive” as used herein is a broad term and is to be given its ordinary and customary meaning to a person of ordinary skill in the art and is not to be limited to a special or customized meaning. The term specifically may refer, without limitation, to an analyte sensor operating outside of the user's body, in particular without penetration and/or penetration of the skin.

The analyte sensor is configured for on-skin wearing. The term “on-skin wearing” as used herein is a broad term and is to be given its ordinary and customary meaning to a person of ordinary skill in the art and is not to be limited to a special or customized meaning. The term specifically may refer, without limitation, to the fact the analyte sensor is mountable and/or attachable to an outer skin surface and/or that the analyte sensor is at least partially in contact with an outer skin surface. The outer skin surface may be the epidermis. For performing an analyte detection, the analyte sensor may be brought in contact with the outer skin surface, e.g., such that the analyte sensor can have direct contact with sweat pores on the epidermis. The analyte sensor may be mountable and/or attachable to an outer skin surface by using at least one patch and/or the analyte sensor may be a part of a patch. The analyte sensor may be skin worn by a user. The analyte sensor may be worn as wristwatch, bracelet, and the like. For example, the analyte sensor may be a patch, firmly contacting the skin surface. The analyte sensor may be an element of a non-invasive monitoring system, as will be described in more detail below. The non-invasive monitoring system may comprise at least one read-out device, e.g. at least one optical read-out device. For example, the read-out device may be a dedicated firmly attached transmitter for high quality continuous read-outs. For example, the read-out device may be or may be comprised by a smart watch, fitness band for continuous monitoring. The read-out device may be an external device for single readouts such as for flash glucose monitoring.

The term “user” as used herein is a broad term and is to be given its ordinary and customary meaning to a person of ordinary skill in the art and is not to be limited to a special or customized meaning. The term specifically may refer, without limitation, to a human being or an animal, independent from the fact that the human being or animal, respectively, may be in a healthy condition or may suffer from one or more diseases. As an example, the user may be a human being or an animal suffering from diabetes. However, additionally or alternatively, the disclosure may be applied to other types of users.

The analyte sensor comprises at least two electrodes. The term “electrode” as used herein is a broad term and is to be given its ordinary and customary meaning to a person of ordinary skill in the art and is not to be limited to a special or customized meaning. The term specifically may refer, without limitation, to a, generally arbitrary shaped, electrical conductor. The analyte sensor may be a two electrode sensor. However, embodiments are possible in which the analyte sensor comprises more than two electrodes. The analyte sensor may comprise more than two electrodes. For example, the analyte sensor may comprise one or more electrodes of the same type.

Each of the electrodes of the analyte sensor may have a skin contacting surface. The term “skin contacting surface” as used herein is a broad term and is to be given its ordinary and customary meaning to a person of ordinary skill in the art and is not to be limited to a special or customized meaning. The term specifically may refer, without limitation, to a surface of the respective electrode configured for contacting the electrode with the skin of the user, either directly or indirectly. For example, the skin contacting surface may be the skin-contacting layer directly in contact with the skin of the user. Alternatively, additional layers between the skin contacting surface and the skin may be used, e.g. the skin contacting surface may be contacted indirectly via at least one additional element such as at least one additional hydrogel layer e.g. for increasing skin compatibility, and the like.

At least one of the electrodes is a detection electrode. The term “detection electrode” as used herein is a broad term and is to be given its ordinary and customary meaning to a person of ordinary skill in the art and is not to be limited to a special or customized meaning. The term specifically may refer, without limitation, to an electrode configured for performing at least one electrochemical detection reaction for detecting the at least one analyte.

The detection electrode may comprise a multi-layer setup.

The detection electrode may comprise a conductive layer comprising one or more of Au, carbon, gold plated copper, or other electrically conductive material, on a substrate. For example, the conductive layer may be one or more of sputtered, screen-printed, or coated on the substrate. For example, the conductive layer may be screen printed, wherein silver-based ink may be applied for forming conductive traces, e.g., as described in Amay J. Bandodkar et al. “Tattoo-Based Noninvasive Glucose Monitoring: A Proof-of-Concept Study”, Anal. Chem. 2015, 87, 394-398, dx.doi.org/10.1021/ac504300n.

The detection electrode comprises at least one analyte-responsive layer. The term “analyte-responsive layer” as used herein is a broad term and is to be given its ordinary and customary meaning to a person of ordinary skill in the art and is not to be limited to a special or customized meaning. The term specifically may refer, without limitation, to a layer configured for exhibiting at least one physical property depending on a quantity of the analyte, e.g., changing at least one chemical and/or physical property depending on the analyte concentration, such as its ionic conductivity, hydrophilicity and/or volume. For example, the analyte may be glucose and the analyte-responsive layer may be a glucose-responsive layer.

The analyte-responsive layer may be an equilibrium analyte-responsive layer. The term “equilibrium analyte-responsive layer” as used herein is a broad term and is to be given its ordinary and customary meaning to a person of ordinary skill in the art and is not to be limited to a special or customized meaning. The term specifically may refer, without limitation, to an analyte-responsive layer comprising an equilibrium within the used detection chemistry. In the analyte-responsive layer, an equilibrium exists between uncharged and charged forms of analyte binding molecules and counter ions. In case of addition of analyte, the charged form binds it, leaving the counter ion. The equilibrium between uncharged and the charged forms will be continuously re-established. The amount of the free counter ions, thus, is proportional to the analyte quantity. The concentration of the free counter ions affects the physico-chemical property of the analyte-responsive layer. For example, the equilibrium may be related to two forms of the boronic acid-based analyte-responsive layer. However, embodiments are thinkable in which the analyte-responsive layer is not an equilibrium analyte-responsive layer but works in a different way.

The non-invasive analyte sensor may be an equilibrium sensor. Thus, it does not actively consume the analyte, e.g., glucose, and can only work under conditions, where the bulk glucose concentration actively changes, e.g., in-vivo. Correspondingly, usually such equilibrium sensor cannot be used for a non-invasive on-skin sensor, which measures glucose levels in the bodily leachates, such as sweat. The reason is slow resorption of glucose by the skin, so that the sensor would show the accumulated glucose concentration, which does not correspond to the actual glucose levels in, e.g., ISF. The present disclosure allows for providing a set-up, where the glucose concentration within the detection volume corresponds to the bulk glucose concentration. The term “equilibrium sensor” as used herein is a broad term and is to be given its ordinary and customary meaning to a person of ordinary skill in the art and is not to be limited to a special or customized meaning. The term specifically may refer, without limitation, to an analyte sensor having an axial equilibrium. In particular, the axial equilibrium relates to the analyte concentration between the bulk concentration, coming with the sample, e.g., leachate, and zero (within tolerances) as the whole analyte is being consumed by the conversion layer. Thus, an axial axis from proximal to distal, the concentration of the analyte drops from the maximum, as delivered by the sample and zero, where it is consumed by the conversion layer. The analyte-responsive layer may be placed somewhere on said axis and it measures a signal proportional to the input at the proximal end. For example, the detection electrode may comprise at its proximal end the analyte-responsive layer and more distal from the skin the conversion layer.

For example, the analyte-responsive layer comprises at least one analyte-responsive hydrogel layer. The term “hydrogel” as used herein is a broad term and is to be given its ordinary and customary meaning to a person of ordinary skill in the art and is not to be limited to a special or customized meaning. The term specifically may refer, without limitation, to a solid material comprising a three-dimensional polymeric matrix or network in the medium of a liquid.

The non-invasive analyte sensor may operate on a detection principle which is based on a glucose-responsive hydrogel (GRH) bearing phenylborate derivatives. However, embodiments of the non-invasive analyte sensor are not limited by use of the phenylborate derivate based GRHs and thus is not limited to diol detection only. Phenylboronic acid and its derivatives are known to form reversible covalent complexes with diol units, such as glucose.

For example, the analyte-responsive polymer gel layer comprises boronic acid. For example, the analyte-responsive polymer gel may comprise a phenylborate. For example, the analyte-responsive polymer gel may comprise 3-(Acrylamido)phenylboronicacid, 4-Vinylpyridine and Divinylbenzene or Acrylamide and 3-acrylamidophenylboronic acid or ethyl acrylate, 3-(Acrylamido)phenylboronic acid, N-vinylpyrrolidone and ethylene glycol dimethacrylate Analyte-responsive polymer gels may generally comprise boronic acid groups or concanavalin A groups (Matthew J. Webber1, 2015) doi: 10.3109/1061186X.2015.1055749.

For example, the analyte-responsive polymer gel may comprise poly(N-isopropylacryl-amide). Analyte-responsive polymer gels are generally known. For example, glucose-responsive polymer gels are described in “Glucose-Responsive Polymer Gel Bearing Phenylborate Derivative as a Glucose-Sensing Moiety Operating at the Physiological pH”, Matsumoto et al., Biomacromolecules 2004, 5, 1038-1045, incorporated herein by reference. Without being bound by theory, phenylboronic acid and its derivatives are known to form reversible covalent complexes with diol units, such as with glucose. Phenylboronic acid compounds in water exist in equilibrium between the uncharged and the charged forms. Only the charged form can make a relatively stable complex with glucose through a reversible covalent bonding, whereas the complex between the uncharged form and glucose is unstable in water due to its high susceptibility to hydrolysis. Because the complex between the charged phenylborate and glucose itself is also anionically charged, the further addition of glucose induces a shift in the equilibrium to the direction of increasing the fraction of the charged forms, and vice versa. Therefore, the introduction of the phenylborate group into an amphiphilic polymer gel structure, such as that of poly(N-isopropylacryl-amide) (PNIPAAm) gel, brings about a reversible volume transition of the gel, which is mainly due to the change in the counterions' osmotic pressure synchronized with the change in the glucose concentration. The analyte-responsive polymer gel may be configured for changing volume and/or charge, in particular continuously, in the presence of the analyte, e.g., glucose. The change in the overall gel charge and volume may lead to the change of the analyte-responsive polymer gels' ionic conductivity, which can be measured, as will be described in more detail below. For example, the analyte sensor may be a glucose sensor comprising boronic acid based glucose-responsive polymer gel. The concentration of glucose can be determined using an excitation signal, e.g., a fast-transient voltage, for the measurement of the ionic conductivity, which is related to the analyte, e.g., glucose, concentration.

For example, in an embodiment the analyte-responsive polymer may have the following composition:

| | | |
|---|---|---|
| 3APBA | 3-(Acrylamido)phenylboronic acid | 5 mol % |
| 4VP | 4-Vinylpyridine | 93 mol % |
| DVB | Divinylbenzene | 1 mol % |
| | Irgacure 2959 | 1 mol % |

The term “conversion layer” as used herein is a broad term and is to be given its ordinary and customary meaning to a person of ordinary skill in the art and is not to be limited to a special or customized meaning. The term specifically may refer, without limitation, to a layer configured for chemically or electrochemically converting the analyte. The conversion layer may actively remove the analyte from the equilibrium analyte-responsive layer, thereby continuously re-creating an equilibrium analyte concentration. The analyte concentration may lie somewhere on the axis between the continuously varying analyte concentration on the leachate and “zero”, where the analyte is completely converted. The equilibrium analyte-responsive layer may be used for analyte detection, but the conversion layer may be required as an additional mechanism, which enable continuous equilibrium re-establishment.

The conversion layer may be designed as a single stack of at least one or a plurality of functional layers. However, other embodiments are thinkable. For example, the conversion layer may comprise two or more laterally distributed functional layers.

The conversion layer may be enzyme-comprising or is non-enzymatic.

For example, the conversion layer may comprise enzyme molecules for converting of the analyte. The conversion layer may comprise glucose oxidase (GOx) or glucose dehydroginase (GDH). In the case of the GOx molecular oxygen from the ambient air can be used as natural electron acceptor. In this case, all layers above the conversion layer may be designed to be at least partially oxygen permeably to supply oxygen to GOx. The reaction product, hydrogen peroxide, may, preferably, be directly decomposed in order to avoid its oxidative action towards enzyme. The conversion layer may comprise some catalyst material, such as manganese oxide or platinum nanoparticles. In the case of GDH, which is an oxygen independent enzyme, another electron acceptor is desired, for instance silver chloride. In order to transfer electrons from the enzyme (co-factor) another mediator may be used. Therefore, the enzyme may be chemically wired by at least one redox hydrogel e.g. at least one Os-complex modified hydrogel.

The conversion layer may be non-enzymatic. For example, the conversion layer is enzymeless, comprising at least one material selected from the material classes of metal-organic frameworks, nanoparticle metal salts of low solubility, like tin-nickel sulfide, graphene oxide, carbon nanotubes and other.

For example, the conversion layer is configured for operating chemically or electrochemically, wherein in the latter case additional polarization voltage is applied against the auxiliary electrode.

The conversion layer may have further functions, e.g. the conversion layer may be used for additional detection of the analyte.

The detection electrode may comprise a stack of at least two functional layers, forming an equilibrium sensor. A proximal functional layer may form the analyte-responsive layer and a distal functional layer may form the conversion layer. The proximal functional layer may be closer to the skin of the user than the distal functional layer. For example, the analyte firstly, passes the analyte-responsive layer, thereby changing its at least one physico-chemical property, and, subsequently, passes towards the conversion layer, where the analyte is chemically or electrochemically converted. The non-invasive analyte sensor may allow for combining an amperometric biosensor and an equilibrium sensor. Both sensors are axially stacked relative to the skin surface at the way, that the bodily leachates, first passes an analyte-responsive layer, changing its conductivity and passes further towards a conversion layer, e.g., an enzymatic layer, where the analyte and, possibly, interferants are electrochemically converted/consumed.

The detection electrode and the auxiliary electrode may be operated as a two (or more)-electrodes potentiostat. The potentiostat may measure electrons generated in the conversion layer, e.g., by an appropriate enzyme, and shuttled by an appropriate mediator from the conversion layer to the conductive layer of the detection electrode and/or by applying a potential to the electrodes for transporting the electrons from the conversion layer to the conductive layer of the detection electrode. If the mediator is intrinsic, e.g., Os-complex modified polymer, it may be a mixture of the mediator and the enzyme, i.e., the enzyme is entrapped in the Os-polymer network. As the enzyme generates electrons, the density thereof may increase in the vicinity of the enzyme. Since the electrons are being distributed statistically in the mediator, effectively there may be an electron transport towards the electrodes surface. This process can be enhanced by applying potential to the electrode. This may result in a classical amperometric detection.

The current, which is flown from the conversion layer to the auxiliary electrode, may be proportional to the analyte concentration. The configuration of the conversion layer and potentiostat may be done to warranty continuous quantitative conversion of the analyte, so that the analyte-responsive layer is always in equilibrium.

The determining of the concentration of the analyte may comprise different electrochemical techniques, such as one or more of electrochemical Impedance Spectroscopy (EIS), potentiometric and amperometric techniques. For example, amperometry may be used for driving the conversion layer. EIS can be used instead or parallel to using a fast-transient voltage, as described below, for analyte detection. Potentiometry can be used additionally to amperometry to elucidate further properties of the measuring system. An additional voltage profile may be added to induce electrophoresis.

In addition, as will be outlined in more detail below, an excitation signal in the form of a fast-transient voltage may be applied to the electrodes for the measurement of the ionic conductivity of the analyte-responsive layer, which is related to the analyte, e.g., glucose, concentration. Since the resistance of the conversion layer is very low, it does not negatively influence the fast-transient measurement thorough the whole stack.

For example, the detection electrode may comprise additional layers between the analyte-responsive layer and the conversion layer. For example, the detection electrode may comprise one or more separation layers such as gels, nets, and the like. For example, the detection electrode may comprise at least one gas permeable water protection layer, e.g., a Teflon layer, on the conversion layer.

For example, the detection electrode may comprise additional gel layers such as the hydrogel, e.g., for increasing skin compatibility, and/or additional membranes, such as micro- or nanoporous membranes, e.g., of PTFE, e.g., used as filters. The detection electrode may be coated with an additional layer of hydrogel on a side facing the skin. The hydrogel may be permeable for the analyte. The hydrogel may act as an adhesive. As outlined above, the analyte-responsive layer may be configured for forming part of the skin contacting surface. However, additional layers between the analyte-responsive layer and the skin may be present.

At least one other of the electrodes is configured as an auxiliary electrode. The term “auxiliary electrode” as used herein is a broad term and is to be given its ordinary and customary meaning to a person of ordinary skill in the art and is not to be limited to a special or customized meaning. The term specifically may refer, without limitation, to a further electrode configured for performing at least one auxiliary function for detection of the analyte by using the detection electrode. The auxiliary electrode is configured as either a counter, or a combined counter-reference electrode. The term “counter electrode” as used herein is a broad term and is to be given its ordinary and customary meaning to a person of ordinary skill in the art and is not to be limited to a special or customized meaning. The term specifically may refer, without limitation, to an electrode configured for performing at least one electrochemical counter reaction adapted for balancing a current flow required by the detection reaction at the detection electrode. The term “reference electrode” as used herein is a broad term and is to be given its ordinary and customary meaning to a person of ordinary skill in the art and is not to be limited to a special or customized meaning. The term specifically may refer, without limitation, to an electrode adapted for providing a constant electrode potential as a reference potential, in particular at least within tolerances, such as by providing a redox system having a constant electrode potential.

The auxiliary electrode may comprise a conductive layer comprising one or more of Au, Ag, carbon, gold, plated copper or other electrically conductive material, on a substrate. The auxiliary electrode may comprise at least one layer comprising one or more of Ag and/or AgCl. The auxiliary electrode may be coated with a hydrogel on a side facing the skin, wherein the hydrogel coating of the auxiliary electrode may contain chloride ions. For example, the auxiliary electrode, e.g., as counter electrode, may comprise a conductive surface, e.g., carbon, gold or silver coated PET or other substrate. The auxiliary electrode may have an additional layer of Ag/AgCl or AgCl only if Ag layer is used as conductive layer, once a combined counter/reference function is desired. On the top of these layers a hydrogel may be coated, which comprises a defined, preferably physiological Cl-concentration for conductivity and reference potential setting of the counter-reference electrode. The hydrogel may also separate silver from skin.

The analyte-responsive layer may be configured for forming part of the skin contacting surface. The detection electrode may be configured for contacting a first skin surface. The auxiliary electrode may be configured for contacting a second skin surface different from the first skin surface. For example, the auxiliary electrode is designed as a concentric ring around the detection electrode.

The non-invasive analyte sensor may comprise a temperature sensor. The temperature may influence diffusional and kinetic processes, or even the equilibrium and needs to be considered by data evaluation.

In a further aspect, a non-invasive continuous monitoring system for on-skin wearing is disclosed. The non-invasive continuous monitoring system comprises at least one non-invasive analyte sensor according to the present disclosure, such as described in one or more of the embodiments enclosed herein, and measurement electronics configured for distributing an excitation signal to the analyte sensor and a reference resistance connected in series with the analyte sensor and measuring at least one voltage distribution over the analyte sensor and the reference resistance during the excitation signal. The measurement electronics is further configured for determining an analyte concentration in the analyte-responsive layer by evaluating the measured voltage distribution.

In a further aspect, a non-invasive continuous monitoring system for on-skin wearing is disclosed. The non-invasive continuous monitoring system comprises at least one non-invasive analyte sensor according to the present disclosure, such as described in one or more of the embodiments enclosed herein, and measurement electronics configured for applying an excitation signal to the analyte sensor and measuring at least one current response. The measurement electronics is further configured for determining an analyte concentration in the analyte-responsive layer by evaluating the measured current response and correlating it with the excitation signal.

For example, the amplitude and frequency of an AC excitation signal is correlated with the amplitude and phase of the response signal.

With respect to definitions and embodiments of the non-invasive analyte sensor reference is made to the description of the non-invasive analyte sensor described in a first aspect or as described in more detail below.

As further used herein, the term “system” refers to an arbitrary set of interacting or interdependent component parts forming a whole. Specifically, the components may interact with each other in order to fulfill at least one common function. The at least two components may be handled independently or may be coupled or connectable. Thus, the term “monitoring system” generally refers to a group of at least two elements or components which are capable of interacting in order to perform at least one analytical detection, specifically at least one analytical detection of at least one analyte of a sample. The monitoring system may be an apparatus, specifically comprising at least two components.

The term “measurement electronics” as used herein is a broad term and is to be given its ordinary and customary meaning to a person of ordinary skill in the art and is not to be limited to a special or customized meaning. The term specifically may refer, without limitation, to an arbitrary electronic device or unit configured for measuring at least one voltage distribution over the analyte sensor and the reference resistance during the excitation signal and for further evaluation.

The measurement electronics may comprise at least one processing device for evaluating the response signal. The term “processing device” as used herein is a broad term and is to be given its ordinary and customary meaning to a person of ordinary skill in the art and is not to be limited to a special or customized meaning. The term specifically may refer, without limitation, to an arbitrary logic circuitry configured for performing basic operations of a computer or system, and/or, generally, to a device which is configured for performing calculations or logic operations. The processing device may be configured for processing basic instructions that drive the computer or system. As an example, the processing device may comprise at least one arithmetic logic unit (ALU), at least one floating-point unit (FPU), such as a math co-processor or a numeric co-processor, a plurality of registers, specifically registers configured for supplying operands to the ALU and storing results of operations, and a memory, such as an L1 and L2 cache memory. The processing device may be a multi-core processor. The processing device may be or may comprise a central processing unit (CPU). Additionally or alternatively, the processing device may be or may comprise a microprocessor, thus specifically the processor's elements may be contained in one single integrated circuitry (IC) chip. Additionally or alternatively, the processing device may be or may comprise one or more application-specific integrated circuits (ASICs) and/or one or more field-programmable gate arrays (FPGAs) and/or one or more tensor processing unit (TPU) and/or one or more chip, such as a dedicated machine learning optimized chip, or the like. The processing device may be configured, such as by software programming, for performing one or more evaluation operations. The processing device may be configured for performing the named step(s). Thus, as an example, the processing device may comprise a software code stored thereon comprising a number of computer instructions. The processing device may provide one or more hardware elements for performing one or more of the indicated operations and/or may provide one or more processors with software running thereon for performing one or more of steps.

As outlined above, the non-invasive analyte sensor may allow for combining an amperometric biosensor and an equilibrium sensor.

For operating the non-invasive analyte sensor as equilibrium sensor, the detection electrode and the auxiliary electrode may be operated as a two (or more)-electrodes potentiostat. The potentiostat may measure electrons generated in the conversion layer, e.g., by an appropriate enzyme, and shuttled by an appropriate mediator from the conversion layer to the conductive layer of the detection electrode and/or by applying a potential to the electrodes for transporting the electrons from the conversion layer to the conductive layer of the detection electrode. As outlined above, if the mediator is intrinsic, e.g., Os-complex modified polymer, it may be a mixture of the mediator and the enzyme, e.g., the enzyme is entrapped in the Os-polymer network. As the enzyme generates electrons, the density thereof may increase in the vicinity of the enzyme. Since the electrons are being distributed statistically in the mediator, effectively there may be an electron transport towards the electrodes surface. This process can be enhanced by applying potential to the electrode. This may result in a classical amperometric detection.

The current may be proportional to the analyte concentration and may be recorded and evaluated into a concentration value by the measurement electronics, e.g., by using at least one predetermined relationship between the analyte concentration and the measured current. The configuration of the conversion layer and potentiostat may be done to warranty continuous quantitative conversion of the analyte, so that the analyte sensor is always in equilibrium.

For operating the non-invasive analyte sensor as amperometric biosensor, the measurement electronics may generate and apply the excitation signal to the electrodes and determines the analyte concentration in the analyte-responsive layer by evaluating a response signal, as will be described in more detail below. In particular, an ionic conductivity measurement of the analyte-responsive layer is used for determining the analyte concentration. Due to high surface area of the auxiliary electrode and relatively low ionic resistivity of the skin, the main portion of the measured resistance is caused by the analyte-responsive layer, which changes its conductivity with analyte concentration. The measurement electronics may be configured for recording impedance values between the two electrodes by applying the excitation signal and determining a response signal.

The advantage of the combination of an equilibrium sensor and an amperometric biosensor may be that the impedimetric measurement is sensitive towards diols, while electrochemical measurement may be distorted by a non-selective oxidation of further electrochemically active substances, e.g., lactate. Thus, having both signals and appropriate use thereof in an algorithm may allow improving the overall selectivity of the measurement system.

The term “excitation signal” as used herein is a broad term and is to be given its ordinary and customary meaning to a person of ordinary skill in the art and is not to be limited to a special or customized meaning. The term specifically may refer, without limitation, to an arbitrary voltage signal. The non-invasive continuous monitoring system, e.g., the measurement electronics, may comprise at least one signal generator device for generating the excitation signal.

The term “signal generator device” as used herein is a broad term and is to be given its ordinary and customary meaning to a person of ordinary skill in the art and is not to be limited to a special or customized meaning. The term specifically may refer, without limitation, to a device, for example a voltage source, being configured to generate a voltage signal. The signal generator device may be and/or may comprise at least one voltage source. The signal generator device may comprise at least one function generator selected from the group consisting of: at least one square wave generator and at least one sine wave generator. The signal generator device may also generate a single pulse which may be unsymmetrically. “Unsymmetrically” in this context means that a first pulse may be different from a second pulse and/or a third pulse and/or any other subsequent pulse. The signal generator device may be part of measurement electronics. The signal generator device may be configured for applying the excitation signal to the electrodes.

The excitation signal may be a fast-transient voltage. The term “fast-transient voltage” as used herein is a broad term and is to be given its ordinary and customary meaning to a person of ordinary skill in the art and is not to be limited to a special or customized meaning. The term specifically may refer, without limitation, to at least one arbitrary voltage signal, in particular an arbitrary voltage change in between two electrodes. The fast-transient voltage may have at least one fast transient signal flank, such as two very steep edges. The fast-transient voltage may comprise a square waveform and/or a sine wave form. For example, the fast-transient voltage may have a square waveform.

The fast-transient voltage may comprise a non-continuous signal such as a pulse. The term “pulse” as used herein is a broad term and is to be given its ordinary and customary meaning to a person of ordinary skill in the art and is not to be limited to a special or customized meaning. The term specifically may refer, without limitation, to a signal having a transient change in the amplitude of the signal from a first value, also denoted baseline value, to a second value, followed by a return to the baseline value or at least approximately to the baseline value. The second value may be a higher or lower value than the baseline value. A pulse duration may be ≤20 μs, more preferably ≤10 μs. The duration of the single pulse must be sufficiently long to be able to record its propagation. The fast-transient voltage may comprise a pulse having two edges: a leading edge or front edge, which is a first edge of the pulse and a trailing edge or back edge, which is a second edge of the pulse.

The term “fast-transient” as used herein is a broad term and is to be given its ordinary and customary meaning to a person of ordinary skill in the art and is not to be limited to a special or customized meaning. The term specifically may refer, without limitation, to time range between first and second values of the signal flank.

The terms first and second “value” may refer to regions or points of the fast-transient voltage, in particular its amplitude. The first value may be the baseline value. The first value may be a local and/or overall minimum of the fast-transient voltage. The first value may be a first plateau of the fast-transient voltage. The first value may refer to a time point with no voltage is applied to the electrodes. The second value may be a local and/or overall extremum of the fast-transient voltage. The second value may be a second plateau of the fast-transient voltage, which may be reached during application of the fast-transient voltage. The second value may be extremum of the fast-transient voltage.

The term “signal flank” as used herein is a broad term and is to be given its ordinary and customary meaning to a person of ordinary skill in the art and is not to be limited to a special or customized meaning. The term specifically may refer, without limitation, to transition of a signal amplitude from low to high signal value or from high to low signal value. The signal flank may be a rising signal flank or a falling signal flank. The signal flank of the fast-transient voltage may have a change in signal from the first value of the signal flank to the second value of the signal flank in a microsecond to nanosecond range. The signal flank may also be referred to as edge. The fast-transient voltage may have a low-to-high transition of a signal amplitude, which is equivalent to rising or positive signal flank, or high-to-low transition of a signal amplitude, which is equivalent to falling or negative signal flank. The fast-transient voltage may have steep edges. Specifically, the fast transition square wave may have a change in voltage from the first value to the second value below or equal 20 ns. The change in voltage from the first value to the second value may be even faster and may be only limited by electronics such as by a fast-transient voltage generator (DAC, DO or others) or a read-out unit (voltage amplifier, ADC, or others). The faster the change of voltage (higher slew rate) and the sharper the transition to the plateau, the more precise the analyte concentration can be determined. The duration of the single fast-transient voltage must be sufficiently long to record the response voltage.

The fast-transient voltage may be applied with a known amplitude. The amplitude of the fast-transient voltage may vary in a broad range and must be optimized for a given set-up. Generally, the lower limit may be limited by the readout technique, which must record the response voltage, mostly by its input range and resolution and may require an additional sufficiently fast voltage amplifier.

The fast-transient voltage may be applied at least once. The fast-transient voltage may be applied after a certain time after contacting the analyte sensor with the skin. The fast-transient voltage may be applied repeatedly, e.g., periodically. The fast-transient voltage may be applied repeatedly, in particular in time intervals from minutes to seconds. A measurement frequency may depend on how often measurement values are required. The measurement electronics may be configured for determining a combined measurement value for reducing a measurement uncertainty. The determining of a combined measurement value may comprise determining of one or more of an average value, a mean value, a median, using a more complex filter such as a Kalman filter. For example, measurement values may be obtained every second and the 60 measurement results may be averaged to a minute-value. The measurement frequency may depend on the application of the analyte sensor.

Additionally, the excitation signal may comprise a continuous variable voltage signal, e.g., having a sine wave signal form. The continuous variable voltage signal may be applied continuously or periodically. The continuous variable voltage signal may be or may comprise a voltage profile, in particular in order to operate the conversion layer.

Additionally, the analyte sensor may comprise at least one pair of electrodes for electrophoresis. The measurement electronics may be configured for applying the additional voltage profile between the pair of electrodes for electrophoresis. The voltage profile may cause electrophoretic effect and accelerate extraction of the bodily leachates.

The measurement electronics is configured for measuring the voltage distribution over the analyte sensor and the reference resistance during the excitation signal. In particular, the measurement electronics is configured for measuring at least one response signal. The term “response signal” as used herein is a broad term and is to be given its ordinary and customary meaning to a person of ordinary skill in the art and is not to be limited to a special or customized meaning. The term specifically may refer, without limitation, to measured propagation of the applied excitation signal. The response signal may be a current or voltage. The response signal may be measured during the duration of the excitation signal. The measurement electronics may be configured for recording at least one impedance value between the electrodes in response to applying the excitation signal.

The response signal may be measured at the reference resistance. The non-invasive continuous monitoring system may comprise at least one reference resistance in series with an ionic resistivity of the analyte-responsive layer. The reference resistance may be selected suitable for determining the ionic resistivity of the analyte-responsive layer, e.g., denoted with Z_{GRH}. A value R_{ref} of the reference resistance may be preferentially in the range of the Z_{GRH}. The reference resistance may relate to a known value such as an average value determined, specifically pre-determined, from a plurality of reference measurements. The reference resistance may reflect the measurement range of the analyte-responsive layer. The reference resistance may reflect required measurement tolerances. During determination of the Z_{GRH} determination, the signal generator device may apply the fast-transient voltage with known amplitude U₁ to the electrodes. Simultaneously a voltage drop U₂ may be measured at the reference resistance R_{ref}. Knowing the amplitude of the applied voltage and the amplitude measured at the R_{ref}, as well as the value of the R_{ref}, Z_{GRH} can be calculated as:

$${Z}_{GRH} = {R}_{ref} \frac{{U}_{2}}{( {U}_{1} - {U}_{2} )}$$

The evaluation of the response signal may be performed by the processing device. The evaluation may further comprise determining the concentration of the analyte from the ionic resistivity of the analyte-responsive layer Z_{GRH} by using at least one pre-determined relationship. For example, the pre-determined relationship may be a linear relationship. For example, the pre-determined relationship may be stored in at least one database of the processing device.

In a further aspect of the present disclosure, a method for determining a concentration of an analyte in a sample using a non-invasive continuous monitoring system according to the present disclosure, such as described in one or more of the embodiments described herein, is disclosed. With respect to definitions and embodiments of the non-invasive continuous monitoring system reference is made to the description of the non-invasive analyte sensor and the non-invasive continuous monitoring system described in a further aspect or as described in more detail below. The method comprises the method steps as given in the corresponding independent claim and as listed as follows. The method steps may be performed in the given order. Further, one or more of the method steps may be performed in parallel and/or in a time overlapping fashion. Further, one or more of the method steps may be performed repeatedly. Further, additional method steps may be present which are not listed.

The method comprising the steps of:
i. contacting the skin contacting surface of the detection electrode and the auxiliary electrode with a skin surface,
ii. distributing an excitation signal to the analyte sensor and a reference resistance connected in series with the analyte sensor and measuring at least one voltage distribution over the analyte sensor and the reference resistance during the excitation signal,
iii. determining the analyte concentration by evaluating the measured voltage distribution.

Additionally, or alternatively, the method comprises the steps of:
I. contacting the skin contacting surface of the detection electrode and the auxiliary electrode with a skin surface,
II. applying an excitation signal to the analyte sensor and measuring at least one current response,
III. determining an analyte concentration in the analyte-responsive layer by evaluating the measured current response and correlating it with the excitation signal.

The term “contacting” as used herein is a broad term and is to be given its ordinary and customary meaning to a person of ordinary skill in the art and is not to be limited to a special or customized meaning. The term specifically may refer, without limitation, to a process of exposing the detection electrode to the fluid, e.g., via attaching and/or mounting the detection electrode to the skin.

The method may comprise measuring a current flow between the conversion layer and the auxiliary electrode and using the measured current flow for additional determining the analyte concentration.

The method may comprise applying at least one additional excitation signal for sample generation and/or extraction, e.g., using electrophoresis.

Further disclosed and proposed herein is a computer program including computer-executable instructions for performing the method according to the present disclosure in one or more of the embodiments enclosed herein when the instructions are executed on a computer or computer network. Specifically, the computer program may be stored on a computer-readable data carrier and/or on a computer-readable storage medium.

As used herein, the terms “computer-readable data carrier” and “computer-readable storage medium” specifically may refer to non-transitory data storage means, such as a hardware storage medium having stored thereon computer-executable instructions. The computer-readable data carrier or storage medium specifically may be or may comprise a storage medium such as a random-access memory (RAM) and/or a read-only memory (ROM).

Thus, specifically, one, more than one or even all of method steps i. to iii. as indicated above may be performed by using a computer or a computer network, preferably by using a computer program. In particular, the computer program may execute and/or trigger executing the method steps ii, iii, II and III.

Further disclosed and proposed herein is a computer program product having program code means, in order to perform the method according to the present disclosure in one or more of the embodiments described herein when the program is executed on a computer or computer network. Specifically, the program code means may be stored on a computer-readable data carrier and/or on a computer-readable storage medium.

Further disclosed and proposed herein is a data carrier having a data structure stored thereon, which, after loading into a computer or computer network, such as into a working memory or main memory of the computer or computer network, may execute the method according to one or more of the embodiments disclosed herein.

Further disclosed and proposed herein is a non-transient computer-readable medium including instructions that, when executed by one or more processors, cause the one or more processors to perform the method according to the present disclosure.

Further disclosed and proposed herein is a computer program product with program code means stored on a machine-readable carrier, in order to perform the method according to one or more of the embodiments disclosed herein, when the program is executed on a computer or computer network. As used herein, a computer program product refers to the program as a tradable product. The product may generally exist in an arbitrary format, such as in a paper format, or on a computer-readable data carrier and/or on a computer-readable storage medium. Specifically, the computer program product may be distributed over a data network.

Finally, disclosed and proposed herein is a modulated data signal which contains instructions readable by a computer system or computer network, for performing the method according to one or more of the embodiments disclosed herein.

Referring to the computer-implemented aspects of the disclosure, one or more of the method steps or even all of the method steps of the method according to one or more of the embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing the samples and/or certain aspects of performing the actual measurements.

Specifically, further disclosed herein are:
a computer or computer network comprising at least one processor, wherein the processor is adapted to perform the method according to one of the embodiments described in this description,
a computer loadable data structure that is adapted to perform the method according to one of the embodiments described in this description while the data structure is being executed on a computer,
a computer program, wherein the computer program is adapted to perform the method according to one of the embodiments described in this description while the program is being executed on a computer,
a computer program comprising program means for performing the method according to one of the embodiments described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method according to one of the embodiments described in this description after having been loaded into a main and/or working storage of a computer or of a computer network, and
a computer program product having program code means, wherein the program code means can be stored or are stored on a storage medium, for performing the method according to one of the embodiments described in this description, if the program code means are executed on a computer or on a computer network.

Summarizing and without excluding further possible embodiments, the following embodiments may be envisaged:

Embodiment 1. A non-invasive analyte sensor comprising at least two electrodes, each having a skin contacting surface, wherein at least one of the electrodes is a detection electrode, wherein the detection electrode comprises at least one analyte-responsive layer and at least one conversion layer, wherein at least one other of the electrodes is configured as an auxiliary electrode configured as either a counter, or a combined counter-reference electrode.

Embodiment 2. The non-invasive analyte sensor according to the preceding embodiment, wherein the detection electrode comprises a stack of at least two functional layers, wherein a proximal functional layer forms the analyte-responsive layer and a distal functional layer forms the conversion layer.

Embodiment 3. The non-invasive analyte sensor according to any one of the preceding embodiments, wherein the analyte-responsive layer is configured for forming part of the skin contacting surface.

Embodiment 4. The non-invasive analyte sensor according to any one of the preceding embodiments, wherein the analyte firstly, passes the analyte-responsive layer, thereby changing its at least one physico-chemical property, and, subsequently, passes towards the conversion layer, where the analyte is chemically or electrochemically converted.

Embodiment 5. The non-invasive analyte sensor according to any one of the preceding embodiments, wherein the analyte-responsive layer comprises at least one analyte-responsive hydrogel layer.

Embodiment 6. The non-invasive analyte sensor according to the preceding embodiment, wherein the analyte-responsive hydrogel layer comprises boronic acid.

Embodiment 7. The non-invasive analyte sensor according to the preceding embodiment, wherein the analyte-responsive hydrogel layer comprises a phenylborate derivative.

Embodiment 8. The non-invasive analyte sensor according to any one of the preceding embodiments, wherein the conversion layer is enzyme-comprising or is non-enzymatic.

Embodiment 9. The non-invasive analyte sensor according to any one of the preceding embodiments, wherein the conversion layer comprises enzyme molecules for converting of the analyte.

Embodiment 10. The non-invasive analyte sensor according to any one of the preceding embodiments, wherein the conversion layer comprises glucose oxidase (GOx) or glucose dehydroginase (GDH).

Embodiment 11. The non-invasive analyte sensor according to any one of the preceding embodiments, wherein the conversion layer is non-enzymatic, comprising at least one material selected from the material classes of metal-organic frameworks, nanoparticle metal salts of low solubility, like tin-nickel sulfide, graphene oxide, carbon nanotubes and other.

Embodiment 12. The non-invasive analyte sensor according to any one of the preceding embodiments, wherein the conversion layer is configured for operating chemically or electrochemically.

Embodiment 13. The non-invasive analyte sensor according to any one of the preceding embodiments, wherein the detection electrode comprises a conductive layer comprising one or more of Au, carbon, gold plated copper or other electrically conductive material on a substrate.

Embodiment 14. The non-invasive analyte sensor according to any one of the preceding embodiments, wherein the detection electrode is coated with a hydrogel on a side facing the skin.

Embodiment 15. The non-invasive analyte sensor according to any one of the preceding embodiments, wherein the auxiliary electrode comprises a conductive layer comprising one or more of Au, Ag, carbon, or gold, plated copper or other electrically conductive material on a substrate.

Embodiment 16. The non-invasive analyte sensor according to any one of the preceding embodiments, wherein the auxiliary electrode comprises at least one layer comprising one or more of Ag and/or AgCl.

Embodiment 17. The non-invasive analyte sensor according to any one of the preceding embodiments, wherein the auxiliary electrode is coated with a hydrogel on a side facing the skin, wherein the hydrogel coating of the auxiliary electrode contains chloride ions.

Embodiment 18. The non-invasive analyte sensor according to any one of the preceding embodiments, wherein the detection electrode is configured for contacting a first skin surface, wherein the auxiliary electrode is configured for contacting a second skin surface different from the first skin surface.

Embodiment 19. The non-invasive analyte sensor according to the preceding embodiment, wherein the auxiliary electrode is designed as a concentric ring around the detection electrode.

Embodiment 20. The non-invasive analyte sensor according to any one of the preceding embodiments, wherein the analyte is glucose.

Embodiment 21. The non-invasive analyte sensor according to any one of the preceding embodiments, wherein the non-invasive analyte sensor is a two-electrode sensor.

Embodiment 22. The non-invasive analyte sensor according to any one of the preceding embodiments, wherein the non-invasive analyte sensor is configured for continuous analyte monitoring.

Embodiment 23. The non-invasive analyte sensor according to any one of the preceding embodiments, wherein the non-invasive analyte sensor is configured for on-skin wearing.

Embodiment 24. The non-invasive analyte sensor according to any one of the preceding embodiments, wherein the non-invasive analyte sensor comprises a temperature sensor.

Embodiment 25. The non-invasive analyte sensor according to any one of the preceding embodiments, wherein the sample is a bodily leachate such as sweat.

Embodiment 26. A non-invasive continuous monitoring system for on-skin wearing comprising at least one non-invasive analyte sensor according to any one of the preceding embodiments and measurement electronics configured for:
distributing an excitation signal to the analyte sensor and a reference resistance connected in series with the analyte sensor and measuring at least one voltage distribution over the analyte sensor and the reference resistance during the excitation signal, wherein the measurement electronics is further configured for determining an analyte concentration in the analyte-responsive layer by evaluating the measured voltage distribution; and/or
applying an excitation signal to the analyte sensor and measuring at least one current response, wherein the measurement electronics is configured for determining an analyte concentration in the analyte-responsive layer by evaluating the measured current response and correlating it with the excitation signal.

Embodiment 27. The non-invasive continuous monitoring system according to the preceding embodiment, wherein the excitation signal is a fast-transient voltage and the voltage distribution is measured as a current or voltage response signal.

Embodiment 28. The non-invasive continuous monitoring system according to the preceding embodiment, wherein the fast-transient voltage comprises a non-continuous signal such as a pulse, wherein a pulse duration is ≤20 μs, preferably ≤10 μs.

Embodiment 29. The non-invasive continuous monitoring system according to any one of the three preceding embodiments, wherein the excitation signal is a continuous variable voltage signal and a response signal is current or voltage.

Embodiment 30. The non-invasive continuous monitoring system according to the preceding embodiment, wherein the voltage distribution is measured during the duration of the excitation signal.

Embodiment 31. The non-invasive continuous monitoring system according to any one of the preceding embodiments referring to a non-invasive continuous monitoring system, wherein the measurement electronics is configured for recording at least one impedance value between the electrodes in response to applying the excitation signal.

Embodiment 32. The non-invasive continuous monitoring system according to any one of the preceding embodiments referring to a non-invasive continuous monitoring system, wherein the measurement electronics is configured for applying an additional voltage profile between the electrodes in order to operate the conversion layer.

Embodiment 33. A method for determining a concentration of an analyte in a sample using a non-invasive continuous monitoring system according to any one of the preceding embodiments referring to a non-invasive continuous monitoring system, the method comprising the steps of:
i. contacting the skin contacting surface of the detection electrode and the auxiliary electrode with a skin surface,
ii. distributing an excitation signal to the analyte sensor and a reference resistance connected in series with the analyte sensor and measuring at least one voltage distribution over the analyte sensor and the reference resistance during the excitation signal,
iii. determining the analyte concentration by evaluating the measured voltage distribution;
   and/or the method comprising the steps of:
I. contacting the skin contacting surface of the detection electrode and the auxiliary electrode with a skin surface,
II. applying an excitation signal to the analyte sensor and measuring at least one current response,
III. determining an analyte concentration in the analyte-responsive layer by evaluating the measured current response and correlating it with the excitation signal.

Embodiment 34. The method according to the preceding embodiment, wherein the method comprises measuring a current flow between the conversion layer and the auxiliary electrode and using the measured current flow for additional determining the analyte concentration.

Embodiment 35. The method according to any one of the preceding embodiments referring to a method, wherein the method comprises applying at least one additional excitation signal for sample generation and/or extraction.

Embodiment 36. A computer program comprising program means for performing the method according to the preceding embodiment while the computer program is being executed on a computer or on a computer network.

### BRIEF DESCRIPTION OF THE DRAWINGS

Further optional features and embodiments will be disclosed in more detail in the subsequent description of embodiments. Therein, the respective optional features may be realized in an isolated fashion as well as in any arbitrary feasible combination, as the skilled person will realize. The scope of the invention is not restricted by the preferred embodiments. The embodiments are schematically depicted in the Figures. Therein, identical reference numbers in these Figures refer to identical or functionally comparable elements.

In the Figures:

FIG. 1 shows an embodiment of a non-invasive continuous monitoring system comprising non-invasive analyte sensor; and

FIG. 2 shows an embodiment of a method for determining a concentration of an analyte in a sample.

### DETAILED DESCRIPTION

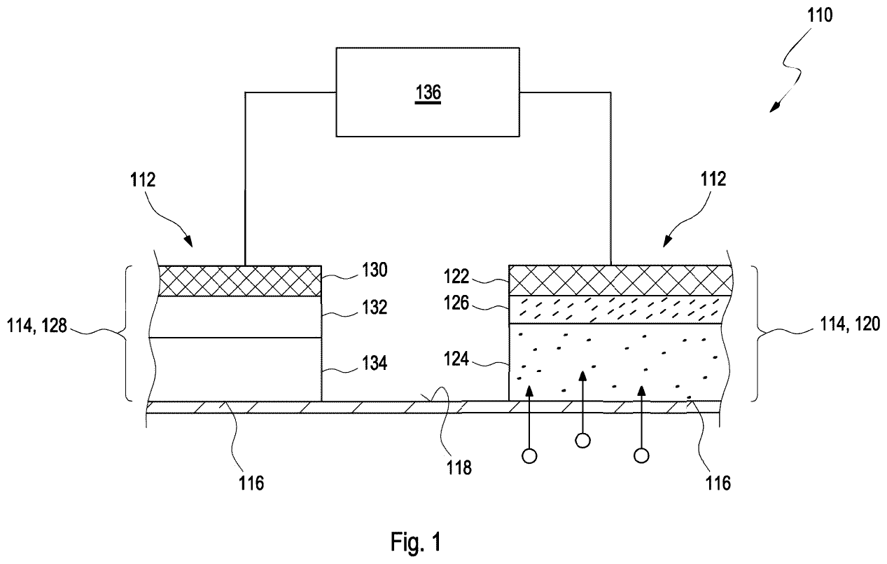

FIG. 1 shows in a highly schematic fashion an exemplary embodiment of a non-invasive continuous monitoring system 110 comprising a non-invasive analyte sensor 112.

The non-invasive analyte sensor 112 comprises at least two electrodes 114. The analyte sensor 112 may be a two electrode sensor. However, embodiments are possible in which the analyte sensor comprises more than two electrodes 114.

Each of the electrodes 114 may have a skin contacting surface 116. The analyte sensor 112 may be mountable and/or attachable to an outer skin surface 118 by using at least one patch and/or the analyte sensor 112 may be a part of a patch. The analyte sensor 112 may be skin worn by a user. The analyte sensor 112 may be worn as wristwatch, bracelet and the like. The skin contacting surface 116 may be configured for contacting the electrode 114 with the skin of a user, either directly or indirectly. For example, the skin contacting surface 116 may be the skin-contacting layer directly in contact with the skin of the user. Alternatively, additional layers between the skin contacting surface 116 and the skin may be used, e.g., the skin contacting surface may be contacted indirectly via at least one additional element such as at least one additional hydrogel layer, e.g., for increasing skin compatibility, and the like.

At least one of the electrodes 114 is a detection electrode 120. The detection electrode 120 may comprise a multi-layer setup.

The detection electrode 120 may comprise a conductive layer 122 comprising one or more of Au, carbon, gold plated copper, or other electrically conductive material, on a substrate. For example, the conductive layer may be one or more of sputtered, screen-printed, or coated on the substrate.

The detection electrode 120 comprises at least one analyte-responsive layer 124. The analyte-responsive layer 124 may be configured for changing at least one chemical and/or physical property depending on the analyte concentration, such as its ionic conductivity, hydrophilicity and/or volume. For example, the analyte may be glucose and the analyte-responsive layer 124 may be a glucose-responsive layer.

The analyte-responsive layer 124 may be an equilibrium analyte-responsive layer. The analyte-responsive layer 124 comprises an equilibrium within the used detection chemistry. In the analyte-responsive layer 124, an equilibrium exists between uncharged and charged forms of analyte binding molecules and counter ions. In case of addition of analyte, the charged form binds it, leaving the counter ion. The equilibrium between uncharged and the charged forms will be continuously re-established. The amount of the free counter ions, thus, is proportional to the analyte quantity. The concentration of the free counter ions affects the physico-chemical property of the analyte-responsive layer. For example, the equilibrium may be related to two forms of the boronic acid-based analyte-responsive layer. However, embodiments are thinkable in which the analyte-responsive layer 124 is not an equilibrium analyte-responsive layer but works in a different way.

The non-invasive analyte sensor 112 may be an equilibrium sensor. Thus, it does not actively consume the analyte, e.g., glucose, and can only work under conditions, where the bulk glucose concentration actively changes, e.g., in-vivo. Correspondingly, usually such equilibrium sensor cannot be used for a non-invasive on-skin sensor, which measures glucose levels in the bodily leachates, such as sweat. The reason is slow resorption of glucose by the skin, so that the sensor would show the accumulated glucose concentration, which does not correspond to the actual glucose levels in, e.g., ISF. The present disclosure allows for providing a set-up, where the glucose concentration within the detection volume corresponds to the bulk glucose concentration. The non-invasive analyte sensor 112 has an axial equilibrium. In particular, the axial equilibrium relates to the analyte concentration between the bulk concentration, coming with the sample, e.g., leachate, and zero (within tolerances) as the whole analyte is being consumed by the conversion layer. Thus, an axial axis from proximal to distal, the concentration of the analyte drops from the maximum, as delivered by the sample and zero, where it is consumed by the conversion layer 126. The analyte-responsive layer 124 may be placed somewhere on said axis and it measures a signal proportional to the input at the proximal end. For example, the detection electrode 120 may comprise at its proximal end the analyte-responsive layer 124 and more distal from the skin the conversion layer 126.

For example, the analyte-responsive layer 124 comprises at least one analyte-responsive hydrogel layer. The non-invasive analyte sensor 112 may operate on a detection principle which uses based on a glucose-responsive hydrogel (GRH) bearing a phenylborate derivatives. However, embodiments of the non-invasive analyte sensor 112 are not limited by using of the phenylborate derivate based GRHs and thus is not limited to diol detection only. Phenylboronic acid and its derivatives are known to form reversible covalent complexes with diol units, such as glucose.

For example, the analyte-responsive hydrogel layer comprises boronic acid. For example, the analyte-responsive hydrogel may comprise a phenylborate. For example, the analyte-responsive hydrogel may comprise 3-(Acrylamido)phenylboronicacid, 4-Vinylpyridine and Divinylbenzene or Acrylamide and 3-acrylamidophenylboronic acid or ethyl acrylate, 3-(Acrylamido)phenylboronic acid, N-vinylpyrrolidone and ethylene glycol dimethacrylate Analyte-responsive hydrogels may generally comprise boronic acid groups or concanavalin A groups (Matthew J. Webber1, 2015) doi: 10.3109/1061186X.2015.1055749.

For example, the analyte-responsive hydrogel may comprise poly(N-isopropylacryl-amide). Analyte-responsive hydrogel are generally known. For example, glucose-responsive hydrogel are described in “Glucose-Responsive Polymer Gel Bearing Phenylborate Derivative as a Glucose-Sensing Moiety Operating at the Physiological pH”, Matsumoto et al., Biomacromolecules 2004, 5, 1038-1045, incorporated herein by reference. Without being bound by theory, phenylboronic acid and its derivatives are known to form reversible covalent complexes with diol units, such as with glucose. Phenylboronic acid compounds in water exist in equilibrium between the uncharged and the charged forms. Only the charged form can make a relatively stable complex with glucose through a reversible covalent bonding, whereas the complex between the uncharged form and glucose is unstable in water due to its high susceptibility to hydrolysis. Because the complex between the charged phenylborate and glucose itself is also anionically charged, the further addition of glucose induces a shift in the equilibrium to the direction of increasing the fraction of the charged forms, and vice versa. Therefore, the introduction of the phenylborate group into an amphiphilic polymer gel structure, such as that of poly(N-isopropylacryl-amide) (PNIPAAm) gel, brings about a reversible volume transition of the gel, which is mainly due to the change in the counterions' osmotic pressure synchronized with the change in the glucose concentration. The analyte-responsive hydrogel may be configured for changing volume and/or charge, in particular continuously, in the presence of the analyte, e.g. glucose. The change in the overall gel charge and volume may lead to the change of the analyte-responsive hydrogels' ionic conductivity, which can be measured, as will be described in more detail below. For example, the analyte sensor may be a glucose sensor comprising boronic acid based glucose-responsive hydrogel gel. The concentration of glucose can be determined using an excitation signal, e.g., a fast-transient voltage, for the measurement of the ionic conductivity, which is related to the analyte, e.g., glucose, concentration.

The detection electrode 120 further comprises at least one conversion layer 126. The conversion layer 126 may be configured for chemically or electrochemically converting the analyte. The analyte-responsive layer 124 may be used for analyte detection, but the conversion layer 126 is required as an additional mechanism, which enable continuous equilibrium re-establishment.

The conversion layer 126 may be enzyme-comprising or be non-enzymatic. For example, the conversion layer 126 may comprise enzyme molecules for converting of the analyte. The conversion layer 126 may comprise glucose oxidase (GOx) or glucose dehydroginase (GDH). For example, the conversion layer 126 is enzymeless, comprising at least one material selected from the material classes of metal-organic frameworks, nanoparticle metal salts of low solubility, like tin-nickel sulfide, graphene oxide, carbon nanotubes and other. For example, the conversion layer 126 is configured for operating chemically or electrochemically, wherein in the latter case additional polarization voltage is applied against an auxiliary electrode 128.

The detection electrode 120 may comprises a stack of at least two functional layers. A proximal functional layer may form the analyte-responsive layer 124 and a distal functional layer may form the conversion layer 126. The proximal functional layer may be closer to the skin of the user than the distal functional layer. For example, the analyte firstly, passes the analyte-responsive layer 124, thereby changing its at least one physico-chemical property, and, subsequently, passes towards the conversion layer 126, where the analyte is chemically or electrochemically converted. The non-invasive analyte sensor 112 may allow for combining an amperometric biosensor and an equilibrium sensor. Both sensors are axially stacked relative to the skin surface at the way, that the bodily leachates, first passes an analyte-responsive layer 124, changing its conductivity and passes further towards a conversion layer 126, e.g., an enzymatic layer, where the analyte and, possibly, interferants are electrochemically converted/consumed.

For example, the detection electrode 120 may comprise additional layers between the analyte-responsive layer 124 and the conversion layer 126. For example, the detection electrode 120 may comprise one or more separation layers such as gels, nets, and the like. For example, the detection electrode 120 may comprise at least one gas permeable water protection layer, e.g., a Teflon layer, on the conversion layer.

For example, the detection electrode 120 may comprise additional gel layers such as the hydrogel, e.g., for increasing skin compatibility, and/or additional membranes, such as micro- or nanoporous membranes, e.g., of PTFE, e.g., used as filters. The detection electrode 120 may be coated with an additional layer of hydrogel on a side facing the skin. The hydrogel may be permeable for the analyte. The hydrogel may act as an adhesive. As outlined above, the analyte-responsive layer 124 may be configured for forming part of the skin contacting surface 116. However, additional layers between the analyte-responsive layer 124 and the skin may be present.

At least one other of the electrodes 114 is configured as an auxiliary electrode 128. The auxiliary electrode 128 is configured as either a counter, or a combined counter-reference electrode. In this embodiment, the auxiliary electrode 128 may comprise a conductive layer 130 comprising carbon. The auxiliary electrode 128 may comprise at least one layer 132 comprising one or more of Ag and/or AgCl. The auxiliary electrode 128 may be coated with a hydrogel 134 on a side facing the skin, wherein the hydrogel coating of the auxiliary electrode may contain chloride ions.

The detection electrode 120 may be configured for contacting a first skin surface. The auxiliary electrode 128 may be configured for contacting a second skin surface different from the first skin surface. For example, the auxiliary electrode 128 is designed as a concentric ring around the detection electrode 120.

The non-invasive continuous monitoring system 110 further comprises measurement electronics 136 configured for distributing an excitation signal to the analyte sensor 112 and a reference resistance connected in series with the analyte sensor 112 and measuring at least one voltage distribution over the analyte sensor 112 and the reference resistance during the excitation signal. The measurement electronics 136 is further configured for determining an analyte concentration in the analyte-responsive layer by evaluating the measured voltage distribution, e.g., a response signal. The measurement electronics 136 may comprise at least one processing device for evaluating the response signal.

The non-invasive continuous monitoring system 110 may combine an amperometric biosensor and an equilibrium sensor.

For operating the non-invasive analyte sensor 112 as equilibrium sensor, the detection electrode 120 and the auxiliary electrode 128 may be operated as a two (or more)-electrodes potentiostat. The potentiostat may measure electrons generated in the conversion layer 126, e.g., by an appropriate enzyme, and shuttled by an appropriate mediator from the conversion layer 126 to the conductive layer 122 of the detection electrode 120 and/or by applying a potential to the electrodes 114 for transporting the electrons from the conversion layer 126 to the conductive layer 122 of the detection electrode 120. If the mediator is intrinsic, e.g., Os-complex modified polymer, it may be a mixture of the mediator and the enzyme, i.e., the enzyme is entrapped in the Os-polymer network. As the enzyme generates electrons, the density thereof may increase in the vicinity of the enzyme. Since the electrons are being distributed statistically in the mediator, effectively there may be an electron transport towards the electrodes surface. This process can be enhanced by applying potential to the electrode. This may result in a classical amperometric detection. The current may be proportional to the analyte concentration and may be recorded and evaluated into a concentration value by the measurement electronics 136, e.g., by using at least one predetermined relationship between the analyte concentration and the measured current.

The configuration of the conversion layer 126 and potentiostat may be done to warranty continuous quantitative conversion of the analyte, so that the analyte-responsive layer 124 is always in equilibrium.

For operating the non-invasive analyte sensor 112 as an amperometric biosensor, the measurement electronics 136 may generate and apply the excitation signal to the electrodes 114 and determine the analyte concentration in the analyte-responsive layer 124 by evaluating a response signal. In particular, an ionic conductivity measurement of the analyte-responsive layer 124 is used for determining the analyte concentration. Due to high surface area of the auxiliary electrode 128 and relatively low ionic resistivity of the skin, the main portion of the measured resistance is caused by the analyte-responsive layer 124, which changes its conductivity with analyte concentration. The measurement electronics 136 may be configured for recording impedance values between the two electrodes 114 by applying the excitation signal and determining a response signal.

The non-invasive continuous monitoring system 110, e.g., the measurement electronics 136, may comprise at least one signal generator device for generating the excitation signal. The excitation signal may be a fast-transient voltage. For example, the fast-transient voltage may have a square waveform. The fast-transient voltage may comprise a non-continuous signal such as a pulse. A pulse duration may be ≤20 μs, more preferably ≤10 μs. The fast-transient voltage may be applied at least once to the electrodes. The fast-transient voltage may be applied after a certain time after contacting the analyte sensor with the skin. The fast-transient voltage may be applied repeatedly to the electrodes, e.g., periodically. The fast-transient voltage may be applied repeatedly to the electrodes, in particular in time intervals from minutes to seconds.

Additionally, the excitation signal may comprise a continuous variable voltage signal, e.g., having a sine wave signal form. The continuous variable voltage signal may be applied continuously or periodically. The continuous variable voltage signal may be or may comprise a voltage profile, in particular in order to operate the conversion layer 126.

Additionally, the analyte sensor 112 may comprise at least one pair of electrodes for electrophoresis. The measurement electronics 136 may be configured for applying the additional voltage profile between the electrodes for electrophoresis. The voltage profile may cause electrophoretic effect and accelerate extraction of the bodily leachates.

The response signal may be a current or voltage. The response signal may be measured during the duration of the excitation signal. The measurement electronics 136 may be configured for recording at least one impedance value between the electrodes in response to applying the excitation signal.

The response signal may be measured at the reference resistance, e.g., which may be an element of the measurement electronics 136. The reference resistance may be in series with an ionic resistivity of the analyte-responsive layer 124. The reference resistance may be selected suitable for determining the ionic resistivity of the analyte-responsive layer, e.g., denoted with Z_{GRH}. A value R_{ref} of the reference resistance may be preferentially in the range of the Z_{GRH}. The reference resistance may relate to a known value such as an average value determined, specifically pre-determined, from a plurality of reference measurements. The reference resistance may reflect the measurement range of the analyte-responsive layer 124. The reference resistance may reflect required measurement tolerances. During determination of the Z_{GRH} determination, the signal generator device may apply the fast-transient voltage with known amplitude U₁ to the electrodes. Simultaneously a voltage drop U₂ may be measured at the reference resistance R_{ref}. Knowing the amplitude of the applied voltage and the amplitude measured at the R_{ref}, as well as the value of the R_{ref}, Z_{GRH} can be calculated as:

$${Z}_{GRH} = {R}_{ref} \frac{{U}_{2}}{( {U}_{1} - {U}_{2} )}$$

The evaluation of the response signal may be performed by the processing device. The evaluation may further comprise determining the concentration of the analyte from the ionic resistivity of the analyte-responsive layer Z_{GRH} by using at least one pre-determined relationship. For example, the pre-determined relationship may be a linear relationship. For example, the pre-determined relationship may be stored in at least one database of the processing device.

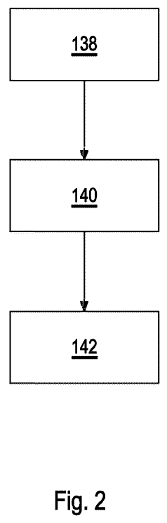

FIG. 2 shows a flowchart of an exemplary embodiment of a method for determining a concentration of an analyte in a sample using a non-invasive continuous monitoring system 110, e.g., as described with respect to FIG. 1. The method steps may be performed in the given order. Further, one or more of the method steps may be performed in parallel and/or in a time overlapping fashion. Further, one or more of the method steps may be performed repeatedly. Further, additional method steps may be present which are not listed. The method comprising the steps of:
i. (138) contacting the skin contacting surface 116 of the detection electrode 120 and the auxiliary electrode 128 with a skin surface 118,
ii. (140) distributing an excitation signal to the analyte sensor 112 and a reference resistance connected in series with the analyte sensor 112 and measuring at least one voltage distribution over the analyte sensor 112 and the reference resistance during the excitation signal,
iii. (142) determining the analyte concentration by evaluating the measured voltage distribution.

The method may comprise measuring a current flow between the conversion layer 126 and the auxiliary electrode 128 and using the measured current flow for additional determining the analyte concentration.

### LIST OF REFERENCE NUMBERS

110 non-invasive continuous monitoring system
112 non-invasive analyte sensor
114 electrode
116 skin contacting surface
118 skin surface
120 detection electrode
122 conductive layer
124 analyte-responsive layer
126 conversion layer
128 auxiliary electrode
130 conductive layer
132 layer
134 hydrogel
136 measurement electronics
138 contacting
140 distributing an excitation signal
142 determining the analyte concentration

While this invention has been described as having an exemplary design, the present invention may be further modified within the spirit and scope of this disclosure. This application is therefore intended to cover any variations, uses, or adaptations of the invention using its general principles.

What is claimed is:

1. A non-invasive analyte sensor comprising:
at least two electrodes, each having a skin contacting surface;
wherein at least one of the electrodes is a detection electrode, wherein the detection electrode comprises at least one analyte-responsive layer and at least one conversion layer;
wherein at least another one of the electrodes is an auxiliary electrode configured as a counter electrode or a combined counter-reference electrode; and
wherein the detection electrode comprises a stack of at least two functional layers, wherein a proximal functional layer forms an equilibrium sensor and a distal functional layer forms the at least one conversion layer.

2. The non-invasive analyte sensor according to claim 1, wherein the at least one analyte-responsive layer comprises at least one analyte-responsive hydrogel layer, wherein the at least one analyte-responsive hydrogel layer comprises boronic acid, wherein the at least one analyte-responsive hydrogel layer comprises a phenylborate derivative.

3. The non-invasive analyte sensor according to claim 1, wherein the at least one conversion layer is enzyme-comprising.

4. The non-invasive analyte sensor according to claim 1, wherein the at least one conversion layer is non-enzymatic.

5. The non-invasive analyte sensor according to claim 1, wherein the at least one conversion layer comprises glucose oxidase (GOx) or glucose dehydroginase (GDH).

6. The non-invasive analyte sensor according to claim 1, wherein the at least one conversion layer is enzymeless and comprises at least one material selected from the material classes of: metal-organic frameworks, nanoparticle metal salts of low solubility, like tin-nickel sulfide, graphene oxide, carbon nanotubes.

7. The non-invasive analyte sensor according to claim 1, wherein the non-invasive analyte sensor is configured to determine a concentration of glucose.

8. The non-invasive analyte sensor according to claim 1, wherein the non-invasive analyte sensor is configured to determine a concentration of an analyte in a sample of a bodily leachate.

9. A non-invasive continuous monitoring system for on-skin wearing comprising:
at least one non-invasive analyte sensor according to claim 1 and measurement electronics configured for:
distributing an excitation signal to the analyte sensor and a reference resistance connected in series with the analyte sensor and measuring at least one voltage distribution over the analyte sensor and the reference resistance during the excitation signal, and determining an analyte concentration in the analyte-responsive layer by evaluating the measured voltage distribution; and/or
applying an excitation signal to the analyte sensor and measuring at least one current response, wherein the measurement electronics is configured for determining an analyte concentration in the analyte-responsive layer by evaluating the measured current response and correlating it with the excitation signal.

10. The non-invasive continuous monitoring system according to claim 9, wherein the excitation signal is a fast-transient voltage and the voltage distribution is measured as a current or voltage response signal, wherein the fast-transient voltage comprises a non-continuous signal.

11. The non-invasive continuous monitoring system according to claim 10, wherein the non-continuous signal is a pulse having duration no greater than 20 μs.

12. The non-invasive continuous monitoring system according to claim 10, wherein the non-continuous signal is a pulse having duration no greater than 10 μs.

13. The non-invasive continuous monitoring system according to claim 9, wherein the measurement electronics is configured for recording at least one impedance value between the at least two electrodes in response to applying the excitation signal.

14. The non-invasive continuous monitoring system according to claim 9, wherein the measurement electronics is configured for applying an additional voltage profile between the at least two electrodes in order to operate the conversion layer by inducing electrophoresis.

15. A method for determining a concentration of an analyte in a sample using a non-invasive continuous monitoring system according to claim 1, the method comprising the steps of:
i. contacting the skin contacting surface of the detection electrode and the auxiliary electrode with a skin surface;
ii. distributing an excitation signal to the analyte sensor and a reference resistance connected in series with the analyte sensor and measuring at least one voltage distribution over the analyte sensor and the reference resistance during the excitation signal; and
iii. determining the analyte concentration by evaluating the measured voltage distribution.

16. The method according to claim 15, wherein the method further comprises measuring a current flow between the conversion layer and the auxiliary electrode and using the measured current flow for determining the analyte concentration.

17. A non-transitory computer readable medium having stored thereon computer executable instructions for performing or triggering the performance of steps ii. and iii. of claim 15.

18. A method for determining a concentration of an analyte in a sample using a non-invasive continuous monitoring system according to claim 1, the method comprising the steps of:
I. contacting the skin contacting surface of the detection electrode and the auxiliary electrode with a skin surface;
II. applying an excitation signal to the analyte sensor and measuring at least one current response; and
III. determining an analyte concentration in the analyte-responsive layer by evaluating the measured current response and correlating it with the excitation signal.

19. The method according to claim 18, wherein the method further comprises measuring a current flow between the conversion layer and the auxiliary electrode and using the measured current flow for determining the analyte concentration.

20. A non-transitory computer readable medium having stored thereon computer executable instructions for performing or triggering the performance of steps II. and III. of claim 18.
